(19) United States
(12) Patent Application Publication
(10) Pub. No.: US 2006/0100231 A1
(43) Pub. Date: May 11, 2006

(54) Amorphous clopidogrel hydrogen sulfate

(75) Inventors: Reddy Bandi Parthasaradhi, Hyderabad (IN); Reddy Kura Rathnakar, Hyderabad (IN); Reddy Rapolu Raji, Hyderabad (IN); Reddy Dasari Muralidhara, Hyderabad (IN)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: Hetero Drugs Limited

(21) Appl. No.: 10/433,210

(22) PCT Filed: Mar. 10, 2003
(86) PCT No.: PCT/IN03/00050

§ 371 Date: May 30, 2003

(51) Int. Cl. C07D 498/02; A61K 31/4743

(52) U.S. Cl. 514/301; 546/114

(57) ABSTRACT

The invention relates to a novel amorphous form of clopidogrel hydrogen sulfate, to processes for its preparation thereof and to a pharmaceutical composition containing it.

### FIELD OF THE INVENTION

The invention relates to a novel amorphous form of clopidogrel hydrogen sulfate, to processes for its preparation thereof and to a pharmaceutical composition containing it.

### BACKGROUND OF THE INVENTION

Clopidogrel hydrogen sulfate, chemically methyl (αS)-α-(2-Chlorophenyl)-6,7-dihydrothieno[3,2-c]pyridine-5(4H)-acetate hydrogen sulfate is a platelet aggregation inhibitor which is described in Eur. Pat. No. 281459. Various methods of synthesis of clopidogrel and its salts are disclosed in U.S. Pat. No. 6,215,005, U.S. Pat. No. 6,180,793, U.S. Pat. No. 5,132,435, U.S. Pat. No. 6,080,875 and WO 02/059128. U.S. Pat. No. 6,429,210 claims a crystalline form of clopidogrel hydrogen sulfate, designated as Form II. The process described in Eur. Pat. No. 281459 for the preparation of clopidogrel hydrogen sulfate leads to a crystalline form which is called Form I.

A novel amorphous form of clopidogrel hydrogen sulfate (hereinafter sometimes referred to as amorphous clopidogrel hydrogen sulfate) has been synthesized and it has been found that it is non-hygroscopic, possesses good dissolution characteristics and adequate stability over the time.

Thus, amorphous clopidogrel hydrogen sulfate is suitable for pharmaceutical formulation as a platelet aggregation inhibitor. The object of the present invention, thus, is to provide a novel amorphous form of clopidogrel hydrogen sulfate, process for preparing it and pharmaceutical formulations containing it.

### BRIEF DESCRIPTION OF THE DRAWINGS

FIG. 1 is a powder x-ray diffractogram of amorphous clopidogrel hydrogen sulfate. Powder x-ray diffraction spectrum was' measured on a Siemens D-5000 diffractometer.

### DESCRIPTION OF THE INVENTION

According to one aspect of the present invention, there is provided clopidogrel hydrogen sulfate in substantially amorphous form.

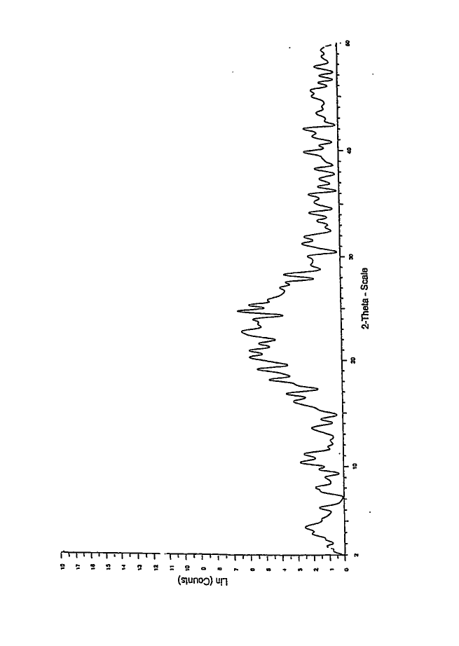

Typical powder x-ray diffraction pattern of amorphous clopidogrel hydrogen sulfate is shown in FIG. 1.

According to another aspect of the present invention, there is provided a process for the preparation of amorphous clopidogrel hydrogen sulfate, which comprises the steps of:
a) dissolving clopidogrel free base in an alcohol;
b) adding conc. sulfuric acid at about 0° C. to about 5° C.;
c) refluxing for about 2 hours;
d) removing the solvent from the solution either by distillation or by vacuum drying or by spray drying.

Alcohol is methanol or ethanol. The solvent can be distilled off from the solution preferably at 40° C.-60° C.

Clopidogrel free base and sulfuric acid are used in the mole ratio of 1:1.

According to another feature of the present invention, there is provided an alternative process for the preparation of amorphous clopidogrel hydrogen sulfate, which comprises the steps of:
a) dissolving clopidogrel hydrogen sulfate in an alcohol;
b) refluxing for about 2 hours;
c) removing the solvent from the solution either by distillation or by vacuum drying or by spray drying.

Alcohol is methanol or ethanol. The solvent can be distilled off from the solution preferably at about 40° C. to about 60° C.

Clopidogrel hydrogen sulfate can be in a crystalline form (Form I or Form II) or in a solvated crystalline form. If solvate is used, the solvent that is the part of clopidogrel hydrogen sulfate solvate is also removed during distillation or dyring. Preferably, clopidogrel hydrogen sulfate is in the form of isopropyl alcohol solvate.

The isopropyl alcohol content of clopidogrel hydrogen sulfate isopropyl alcohol solvate is preferably between 6.8 to 9.5% mass/mass. Clopidogrel hydrogen sulfate isopropyl alcohol solvate can be prepared by adjusting the pH of the aqueous solution of clopidogrel R-camphor sulfonate to 9-9.5 with saturated aqueous solution of sodium bicarbonate, extracting with ethyl acetate, distilling off the solvent from the organic layer under vacuum, taking the residue in isopropyl alcohol, adding sulfuric acid, refluxing the contents for about 1 hour and separating the crystals at 25-35° C.

According to another feature of the present invention, there is provided a pharmaceutical composition comprising amorphous clopidogrel hydrogen sulfate and a pharmaceutically acceptable carrier. The compositions containing amorphous clopidogrel hydrogen sulfate may be in a form suitable for oral dosage as a tablet, capsule or suspension. Any conventional technique may be used for the preparation of pharmaceutical formulation.

The following non-limiting examples illustrate the invention.

### EXAMPLE 1

Clopidogrel free base (20 gm, 0.0621 mole) is dissolved in ethanol (100 ml). The solution is cooled to 0° C. to 5° C. and conc. sulfuric acid (3.5 ml) is slowly added at this temperature. The solution is heated to reflux and refluxed for 2 hours. The solvent is distilled off completely at 45° C. to 55° C. to give 26.09 gm of amorphous clopidogrel hydrogen sulfate.

### EXAMPLE 2

Example 1 is repeated except that instead of distilling off the solvent, the solution is subjected to vacuum drying to give amorphous clopidogrel hydrogen sulfate.

### EXAMPLE 3

Example 1 is repeated except that instead of distilling off the solvent, the solution is subjected to spray drying to give amorphous clopidogrel hydrogen sulfate.

### EXAMPLE 4

Clopidogrel hydrogen sulfate isopropyl alcohol solvate (50 gm) is dissolved in ethanol (250 ml) and the solution is refluxed for 1 hour. The solvent is distilled off completely at 45° C. to 55° C. to give 46.3 gm of amorphous clopidogrel hydrogen sulfate.

### EXAMPLE 5

Example 4 is repeated except that instead of distilling off the solvent, the solution is subjected to vacuum drying to give 46.2 gm of amorphous clopidogrel hydrogen sulfate.

### EXAMPLE 6

Clopidogrel hydrogen sulfate crystalline form I (20 gm) is dissolved in ethanol (120 ml) and the solution is refluxed for 1 hour. The solvent is distilled off completely at 45° C. to 55° C. to give amorphous clopidogrel hydrogen sulfate in quantitative yield.

### EXAMPLE 7

Example 6 is repeated using clopidogrel hydrogen sulfate crystalline form II instead of clopidogrel hydrogen sulfate Form I to give amorphous clopidogrel hydrogen sulfate in quantitative yield.

1. Clopidogrel hydrogen sulfate in amorphous form.

2. Amorphous clopidogrel hydrogen sulfate as defined in claim 1, further characterized by a powder x-ray diffraction pattern of FIG. 1.

3. A process for preparation of amorphous clopidogrel hydrogen sulfate as defined in claim 1, which comprises the steps of:
a) dissolving clopidogrel base in methanol or ethanol, or mixture thereof;
b) adding conc. sulfuric acid at about 0° C. to about 5° C.;
c) refluxing for about 2 hours; and
d) removing the solvent from the solution either by distillation or by vacuum drying or by spray drying.

4. A process according to claim 3, wherein the solvent is ethanol

5. A process according to claim 3, wherein the solvent is removed by distillation.

6. A process for preparation of amorphous clopidogrel hydrogen sulfate as defined in claim 1, which comprises the steps of:
a) dissolving clopidogrel hydrogen sulfate crystalline form in an alcohol;
b) refluxing for about 2 hours; and
c) removing the solvent from the solution either by distillation or by vacuum drying or by spray drying;
wherein alcohol is methanol or ethanol.

7. A process according to claim 6, wherein clopidogrel hydrogen sulfate crystalline form is Form I.

8. A process according to claim 6, wherein clopidogrel hydrogen sulfate crystalline form is Form II.

9. A process according to claim 6, wherein the solvent is ethanol.

10. A process for preparation of amorphous clopidogrel hydrogen sulfate as defined in claim 1, which comprises the steps of:
a) dissolving clopidogrel hydrogen sulfate solvate in an alcohol;
b) refluxing for about 2 hours;
c) removing the solvents from the solution either by distillation or by vacuum drying or by spray drying;
wherein alcohol is methanol or ethanol.

11. A process according to claim 10, wherein clopidogrel hydrogen sulfate solvate is clopidogrel hydrogen sulfate isopropyl alcohol solvate.

12. A process according to claim 10, wherein alcohol is ethanol.

13. A process according to claim 10, wherein solvents are removed by distillation.

14. A pharmaceutical composition comprising amorphous clopidogrel hydrogen sulfate as defined in claim 1 and a pharmaceutically acceptable carrier.

15. A process according to claim 7, wherein the solvent is ethanol.

16. A process according to claim 8, wherein the solvent is ethanol.
